(19) United States
(12) Patent Application Publication
(10) Pub. No.: US 2005/0079228 A1
(43) Pub. Date: Apr. 14, 2005

(54) Clear, stable topical compositions of clarithromycin and processes for their preparation

(76) Inventors: Ashish Jaiswal, Indore (IN); Mukesh Kumar Garg, Jagraon (IN); Vinod Kumar Arora, New Delhi (IN)

Correspondence Address: Jayadeep R. Deshmukh, Esq.;Ranbaxy, Inc., Suite 2100, 600 College Road East, Princeton, NJ, 08540, US

(21) Appl. No.: 10/856,662

(22) Filed: May 28, 2004

(30) Foreign Application Priority Data
May 30, 2003 (IN) IN 763/DEL/2003

(51) Int. Cl. A61K 31/704; A61K 33/32

(52) U.S. Cl. 424/641; 514/35; 514/57

(57) ABSTRACT

The invention relates to clear, stable topical compositions of clarithromycin for the treatment of acne and processes for their preparation. The transparent topical compositions include clarithromycin, a zinc salt, a pharmaceutically acceptable vehicle and may include gelling agents.

### TECHNICAL FIELD OF THE INVENTION

The technical field of the invention relates to clear, stable topical compositions of clarithromycin for the treatment of acne and processes for their preparation.

### BACKGROUND OF THE INVENTION

Acne vulgaris is an inflammatory disease of the sebaceous glands characterized by comedones (blackheads), papules, pustules, cysts, nodules, and often scars, that appear on the most visible areas of the skin (e.g., the face, chest, back, neck and upper arms). There is an excessive production of sebum during adolescence and puberty. The condition may worsen by a simultaneous increase in the rate of keratinization of the skin's horny layer (the stratum corneum). As the horny cells proliferate, they can form an occlusive plug or comedone. Eventually, the plugged follicles rupture and allow the discharge of their contents, causing local swelling and inflammation. The exposed follicles may darken from the deposition of pigment from damaged cells in the deeper layer of skin. In severe cases, acne can lead to hospitalization of the patient, extensive discomfort, and long term scarring of the skin. Various topical agents are utilized in the treatment of acne, including sulfur, resorcinol, salicylic acid, benzoyl peroxide, retinoids and topical antibiotics, such as tetracyclines, erythromycin, clindamycin and the like.

The search for improved acne treatments has been widespread and continuous during the past several decades. Enhanced cosmetic properties to encourage user compliance, the use of topical therapies in place of systemic drugs to reduce toxicity and side effects, and the introduction of new drugs and formulations represent the forefront of acne treatment advances.

A number of topical preparations of erythromycin have been disclosed for the treatment of acne. These include U.S. Pat. No. 4,132,781 which discloses compositions for the treatment of signs and symptoms of acne. The compositions include from about 0.1 to about 10 percent by weight of an antibiotic of the erythromycin family together with from about 5 to about 99.9 percent by weight of a compound selected from the group consisting of 2-pyrrolidone and an N-lower alkyl-2-pyrrolidone.

Zinc salts have also been taught to be effective in the topical treatment of acne. For example, U.S. Pat. No. 4,261,982 discloses a process for preparing zinc erythromycin, useful in the topical treatment of skin disorders such as acne, by admixing zinc salts with erythromycin base. The patent discloses the percutaneous penetration of zinc salts being greatly enhanced in the presence of erythromycin.

U.S. Pat. No. 4,299,829 describes compositions for topical application in the treatment of skin disorders and dermatoses of bacterial origin. The compositions include a minor proportion of an antibiotic agent selected from erythromycin and its derivatives and a carrier that includes a penetration enhancing amount of diisopropyl sebacate and alcohol.

U.S. Pat. No. 5,455,037 describes stable topical cream compositions for treating dermal microbial infections in animals. The compositions include 3-4% erythromycin, a polysiloxane, ethanol, water and an emulsifier.

Although all of these patents describe topical preparations of erythromycin and their salts and derivatives, none of them are directed to erythromycin derivatives such as clarithromycin.

U.S. Pat. No. 4,331,803 discloses that clarithromycin is a macrolide antibiotic which has strong antibacterial activity against gram-positive bacteria and is generally considered to have more potent antibacterial activity than the parent drug erythromycin.

Recent trends in consumer buying have shifted in its emphasis to a demand for clear products. Consumer items such as shampoos, soaps, moisturizing gels, sunscreens, bath oils, deodorants, and dentifrices are commonly available in clear preparations. It has generally been observed that for topical applications, the consumer usually finds a clear, transparent product to be of a greater aesthetic appeal, in comparison to an opaque or translucent one. Therefore the demand for clear products is likely to continue.

For a composition to be topically effective, cutaneous absorption and penetration through the skin layers are the important factors to be considered. However, the poor solubility profile of clarithromycin presents a technical hindrance in the preparation of a clear, stable formulation which is also topically effective.

U.S. Pat. No. 4,621,075 describes topical pharmaceutical compositions in the form of physically stable gels containing clindamycin phosphate, a zinc fatty acid and a non-aqueous vehicle. Although the composition disclosed in this patent was formed without the use of any conventional gelling agent, there is no mention of a complexation process.

### SUMMARY OF THE INVENTION

In one general aspect there is provided transparent topical compositions of clarithromycin and a zinc salt. The topical compositions include a pharmaceutically acceptable vehicle and may include gelling agents.

Embodiments of the compositions may include one or more of the following features. For example, the clarithromycin may be present at from about 0.1% w/w to about 10% w/w of the formulation. The composition is preferably stable.

The zinc salt may be selected from the group consisting of zinc acetate, zinc propionate, zinc butyrate, zinc pentanoate, zinc hexanoate, zinc heptanoate, zinc decanoate, zinc citrate, zinc maleate, zinc benzoate, zinc chloride, zinc sulfate, zinc phosphate, zinc bromide, zinc salts of amino acid like zinc alanine, zinc methionine and zinc glycine. The clarithromycin and zinc salt may be present in the molar ratio of about 1:0.2 to about 1:2 and may form a complex. More particularly, the clarithromycin and the zinc salt may be combined in the molar ratio of about 1:1 to about 1:1.5.

The pharmaceutically acceptable vehicle may be non-aqueous or hydroalcoholic material. Non-aqueous materials may be selected, for example, from one or more of methanol, ethanol, n-propanol, isopropanol, butanol, propylene glycol, polypropylene glycol, polyethylene glycol, hydrocarbon oils and waxes, lanolin and lanolin derivatives, diisopropyl sebacate, isopropyl myristate, methyl laurate, silicon oil, glycerin, caprylic acid esters, transcutol, labrasol, labrafac, labrafil and mixtures thereof.

The composition may include gelling agents including cellulose ethers such as carbomers, hydroxypropyl cellulose, hydroxymethyl cellulose, hydroxypropyl methyl cellulose, carboxy methyl cellulose, sodium carboxy methyl cellulose, hydroxycellulose; vinyl alcohols; vinyl pyrrolidones; natural gums such as karaya gum, locust bean gum, guar gum, gelan gum, xanthan gum, gum arabic, tragacanth gum, carrageenan, pectin, agar, alginic acid, sodium alginate and the like; methacrylates and polyacrylates and polyoxyethylene-polyoxypropylene copolymers (poloxamers).

The composition may also include pharmaceutically acceptable excipients, including antioxidants, preservatives, pH modifying agent, perfumes, skin penetration enhancers and stabilizers. The composition may be in the pH range from about 6.0 to about 8.0 and may be adjusted using pH modifying agents including organic aliphatic polyhydroxy carboxylic acids, and basic amines including triethanolamine, diethanolamine, diethyl amine, sodium hydroxide, 2 amino-2 methyl-1 propanol, and tris buffer. The concentration of the pH modifying agent may be between about 0.15-2% w/w of the final composition.

The skin penetration enhancers may be selected from one or more of alcohols, short chain alcohols, polyalcohols, amino acids, fatty acids and their esters, azone and azone-like compounds, surfactants, and bile salts.

In another general aspect there is provided a process for preparing a topical composition of clarithromycin. The process includes the steps of dissolving a zinc salt in the presence of a suitable vehicle to form a solution; dispersing clarithromycin in the solution; mixing the solution to form a transparent solution; and, optionally dispersing a gelling agent in the solution.

Embodiments of the process may include one or more of the following features or those described above. For example, the process may include mixing the composition to form a clarithromycin zinc complex. The process may also include the addition of one or more excipients including antioxidants, preservatives, pH modifying agent, perfumes, skin penetration enhancers and stabilizers. The process may also include mixing the composition until all the components of the composition are fully dissolved. The clarithromycin and the zinc salt may be combined in a molar ratio of about 1:0.2 to about 1:2. The topical vehicle may be a non aqueous or a hydroalcoholic material. The pH of the final composition may be between about 6.0 and about 8.0. The composition may be colorless and stable.

According to yet another aspect there is provided a method of treating acne. The method includes administering a transparent topical composition of clarithromycin, a zinc salt, a pharmaceutically acceptable topical vehicle, and optionally one or more gelling agents.

Embodiments of the method may include one or more of the following features or those described above. For example, the administered composition may include clarithromycin and zinc that are complexed. The composition may be colorless and stable.

The details of one or more embodiments of the inventions are set forth in the description below. Other features, objects and advantages of the inventions will be apparent from the description and claims.

### DESCRIPTION OF THE DRAWINGS

FIG. 1 is an infrared (IR) spectroscopy plot of a clarithromycin-zinc acetate treated complex, clarithromycin alone, zinc acetate alone and the physical mixture of clarithromycin and zinc acetate.

FIGS. 2 and 3 are differential scanning calorimetry (DSC) plots of the melting point endotherms of the composition as obtained with the complex and for the physical mixture of clarithromycin and zinc acetate, respectively.

FIGS. 4 and 5 are NMR spectroscopy plots illustrating the chemical shift values obtained with the complex and for clarithromycin alone, respectively.

FIG. 6 is an X-Ray Powder Diffraction (XRD) plot showing the diffraction patterns obtained with the complex and for the physical mixture of clarithromycin and zinc acetate.

### DETAILED DESCRIPTION OF THE INVENTION

The topical compositions described herein include clarithromycin and a zinc salt in a pharmaceutically acceptable topical vehicle. The topical composition may be formulated as gels, solutions or lotions. The pharmaceutically acceptable topical vehicle may be a non-aqueous or a hydroalcoholic material. The topical compositions are preferably transparent, colorless, and exhibit good stability.

Stable, for the purposes of this patent, refers to the chemical and physical stability of a packaged composition. A stable composition is one that maintains its physical appearance, viscosity, and assay after three months of accelerated stability conditions at 40±2° C. and 75±5% relative humidity

The concentration of clarithromycin in the topical composition varies depending on many factors, including the particular condition to be treated, severity of the condition, and other like factors. Accordingly, formulations of different strengths may be formulated containing about 0.1% to about 10.0% w/w clarithromycin of the total weight of the formulation.

Zinc salts may be selected from any of the pharmaceutically acceptable salts such as zinc acetate, zinc propionate, zinc butyrate, zinc pentanoate, zinc hexanoate, zinc heptanoate, zinc decanoate, zinc citrate, zinc maleate, zinc benzoate, zinc chloride, zinc sulfate, zinc phosphate, zinc bromide, zinc salts of amino acids like zinc alanine, zinc methionine, zinc glycine and the like.

Clarithromycin and the zinc salt are preferably combined in a molar ratio ranging from about 1:0.2 to about 1:2. This ratio permits the occurrence of effective complexation of the zinc and clarithromycin. While not intending to be limited by theory, it has been found that such a complexation increases the solubility of clarithromycin in a non-aqueous media, thus facilitating formation of a clear, cosmetically elegant, stable composition.

The pharmaceutically acceptable vehicle may be a non-aqueous or a hydroalcoholic material. Non-aqueous vehicles may be selected, for example, from one or more of methanol, ethanol, n-propanol, isopropanol, butanol, propylene glycol, polypropylene glycol, polyethylene glycol, hydrocarbon oils and waxes, lanolin and lanolin derivatives, diisopropyl sebacate, isopropyl myristate, methyl laurate, silicon oil, glycerin, caprylic acid esters, transcutol, labrasol, labrafac, labrafil and mixtures thereof. Alternatively, hydroalcoholic vehicles may be used. Hydroalcoholic vehicles may increase the cost-effectiveness as compared to the use of non-aqueous vehicles. The solubility of zinc salts in combination with clarithromycin may be enhanced in hydroalcoholic vehicles by the addition of a small amount water soluble alpha-hydroxy or polycarboxylic acids such as citric acid, lactic acid, malonic acid, maleic acid and gentisic acid.

The optional gelling agents may be selected from a wide variety of suitable gelling agents. Gelling agents may include, for example, one or more of cellulose ethers such as hydroxypropyl cellulose, hydroxymethyl cellulose, hydroxypropyl methyl cellulose, carboxy methyl cellulose, sodium carboxy methyl cellulose, hydroxycellulose and the like; vinyl alcohols; vinyl pyrrolidones; natural gums such as karaya gum, locust bean gum, guar gum, gelan gum, xanthan gum, gum arabic, tragacanth gum, carrageenan, pectin, agar, alginic acid, sodium alginate and the like; methacrylates such as those available under the trade name Eudragit® from Rohm Pharma and polyacrylates such as those available under the brand name Carbopol® from B.F. Goodrich. Other gelling agents include polyoxyethylene-polyoxypropylene copolymers (poloxamers) such as those available under the trade name Lutrol®, and the like.

The topical composition may also include other pharmaceutically acceptable excipients including, but not limited to, one or more of preservatives, antioxidants, fragrances or perfumes, skin penetration enhancers and chelating agents.

Antioxidants are used to protect the ingredients of the composition from oxidizing agents that may be included within or come in contact with the composition. Suitable antioxidants can be either water-soluble or oil-soluble. Examples of antioxidants include one or more of water-soluble antioxidants such as ascorbic acid, sodium sulfite, metabisulfite, sodium miosulfite, sodium formaldehyde, sulfoxylate, isoascorbic acid, isoascorbic acid, cysteine hydrochloride, 1,4-diazobicyclo-(2,2,2)-octane, and mixtures thereof. Examples of oil-soluble antioxidants include one or more of ascorbyl palmitate, butytlated hydroxyanisole, butylated hydroxytoluene, potassium propyl gallate, octyl gallate, dodecyl gallate, phenyl-.alpha.-napthyl-amine, and tocopherols such as α-tocopherol.

Suitable preservatives may also be added to the topical composition. Preservative are particularly useful when the composition is to be applied to an area prone to microbial infection, e.g., by bacteria, fungal, or protozoa. Examples of such agents include one or more of benzyl alcohol, chlorobutanol, phenylethyl alcohol, phenylmercuric acetate, potassium sorbate, and sorbic acid, benzoic acid, butyl paraben, ethyl paraben, methyl paraben, propyl pareben, sodium benzoate, phenoxyethanol, ethanol and isopropyl alcohol.

Suitable penetration enhancers may also be added to the topical composition. Examples include one or more of alcohols such as short chain alcohols and polyalcohols; amino acids; essential oils; fatty acids and their esters; azone and azone like compounds; surfactants; bile salts and the like. The penetration enhancers may be added separately or as combinations. A combination of enhancers from different groups may prove effective.

A suitable pH of the topical composition is generally in the range of from about 6.0 to about 8.0, and in particular between about 6.2 to about 7.5. The pH can be adjusted by the use of suitable pH-modifying agents. pH-modifying agents may be selected, for example, from one or more of the group consisting of lactic acid, malic acid, citric acid and other such aliphatic polyhydroxy carboxylic acids, basic amines, such as triethanolamine, diethanolamine, diethyl amine, sodium hydroxide, 2 amino-2 methyl-1 propanol, tris buffer.

Fragrances and perfumes may also be added to the topical composition. Examples include one or more of peppermint, rose oil, rose water, aloe vera, clove oil, menthol, camphor, eucalyptus oil, and other plant extracts.

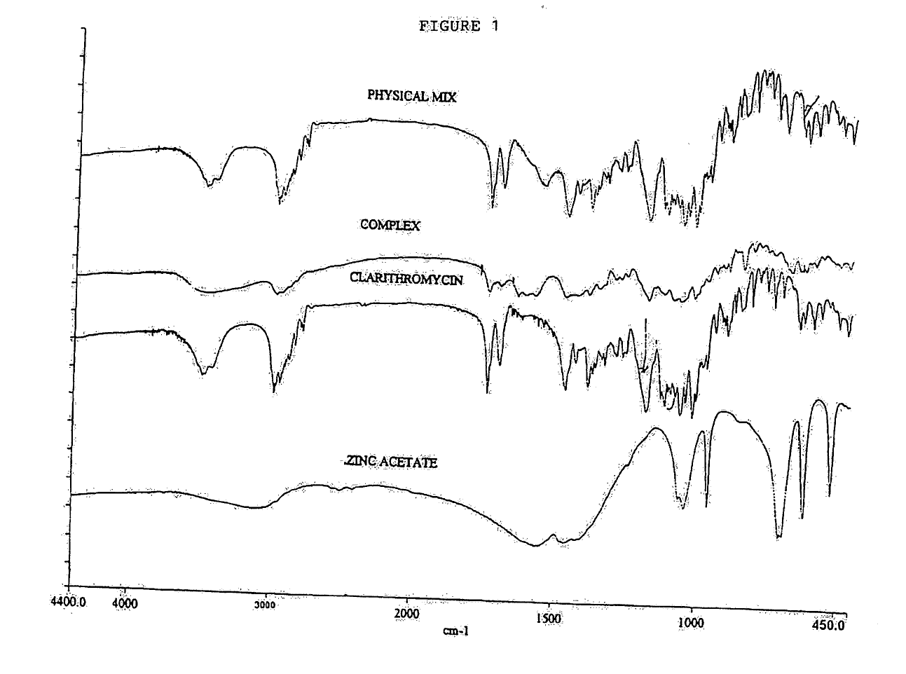

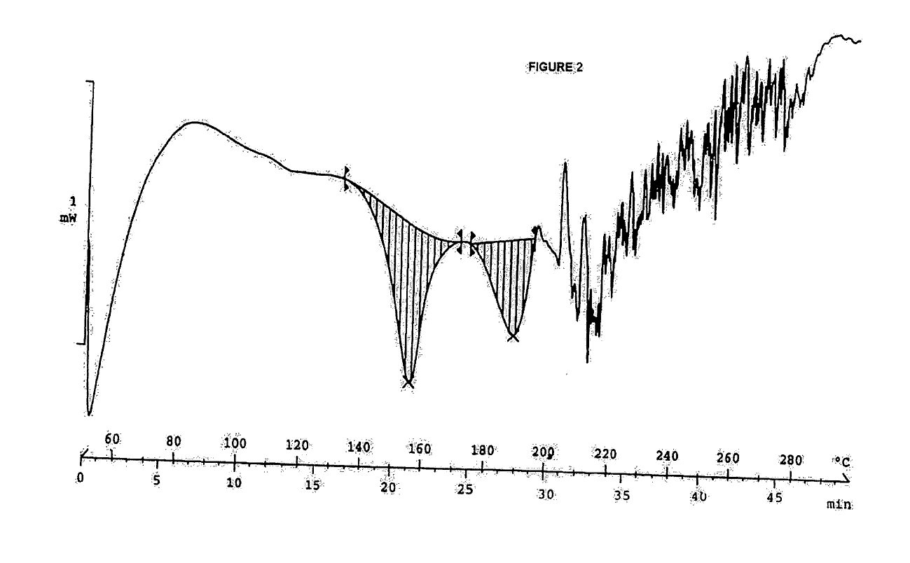

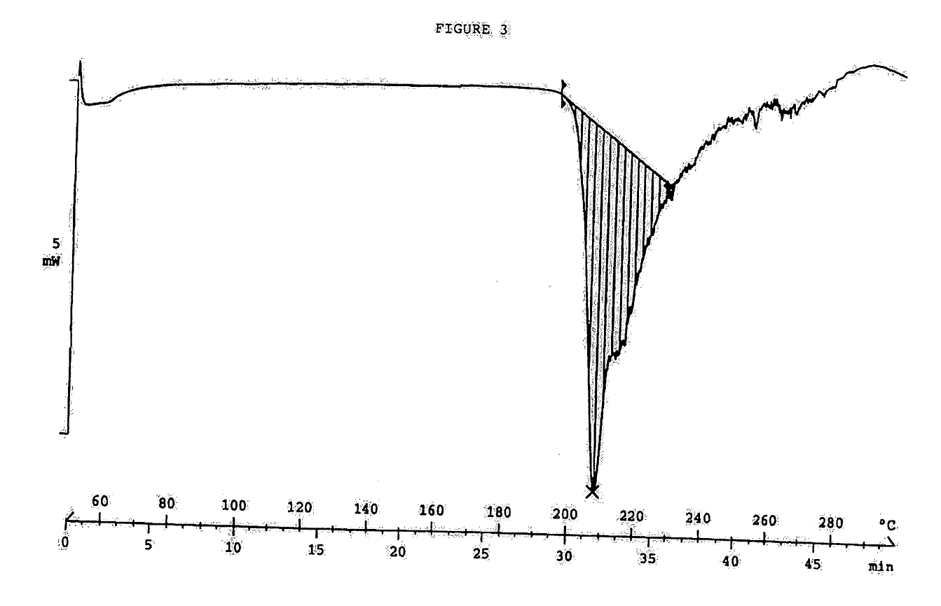

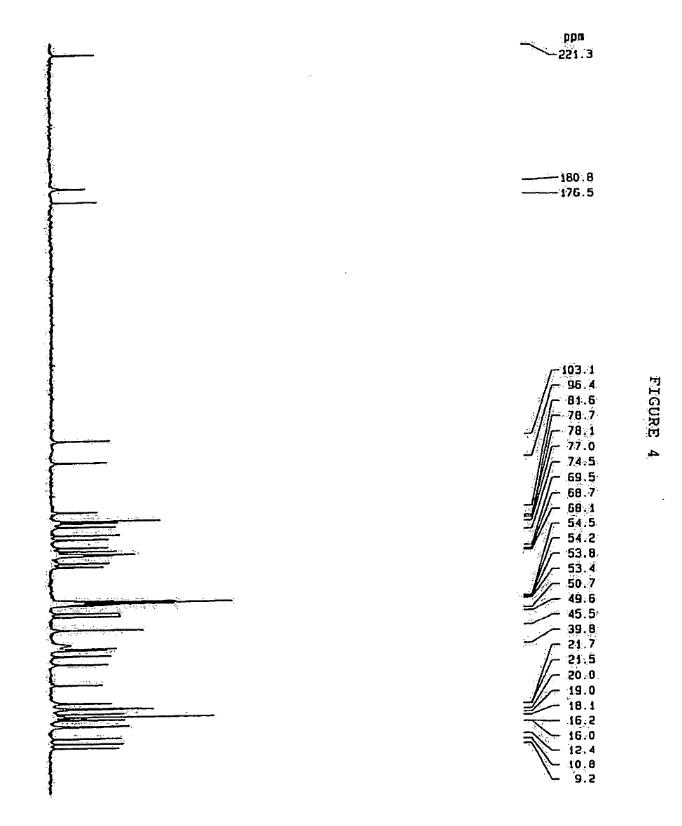

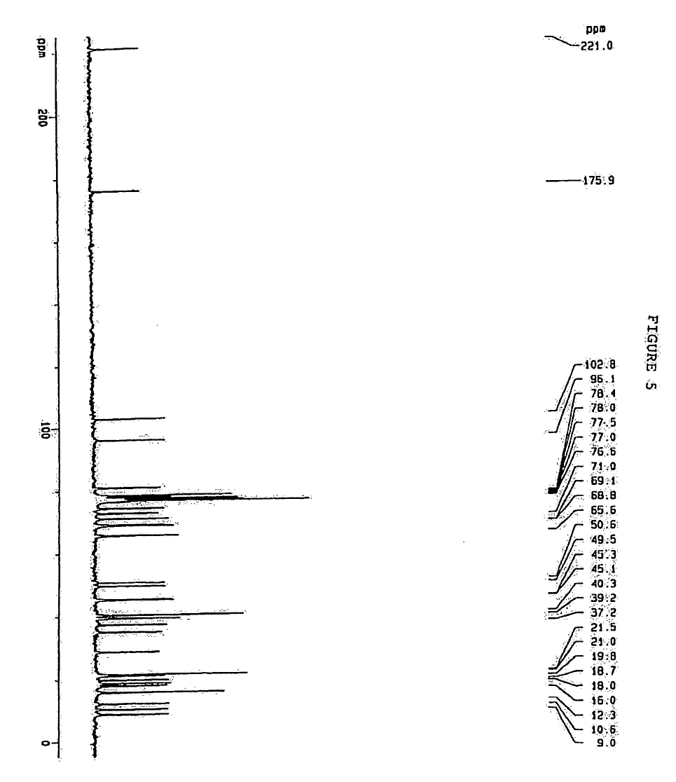

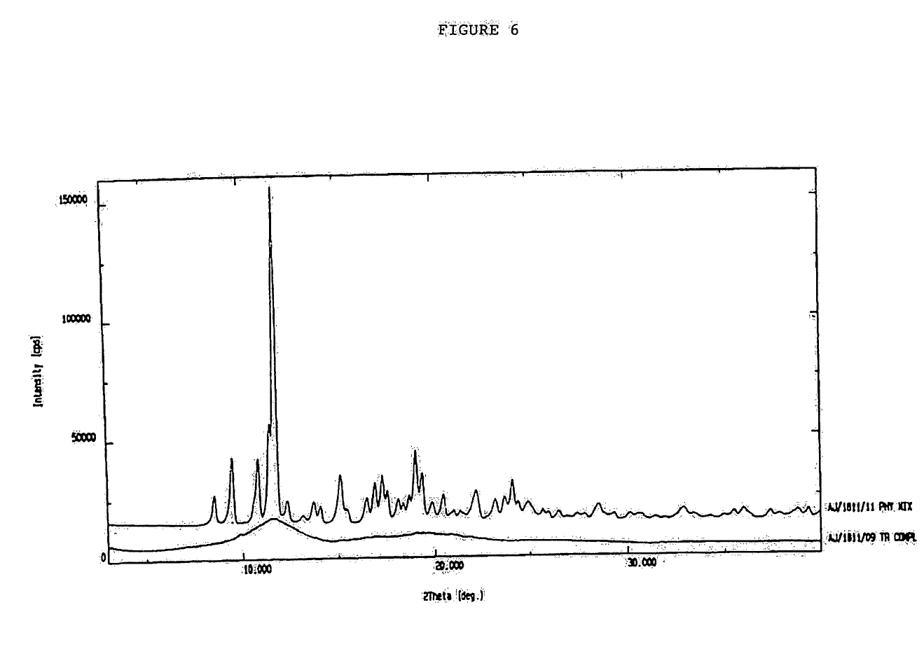

The following techniques were employed to characterize the clarithromycin-zinc complex:
1) IR Spectroscopy: The location of peaks in the fingerprint region of infra red spectrum of clarithromycin-zinc acetate treated complex, clarithromycin alone, zinc acetate alone and the physical mixture of clarithromycin and zinc acetate are shown in FIG. 1.
2) Differential Scanning Calorimetry (DSC): The melting point endotherms of the composition as obtained with the complex and for the physical mixture of clarithromycin and zinc acetate are shown in FIG. 2 and FIG. 3.
3) NMR Spectroscopy: The chemical shift values obtained with the complex and for clarithromycin alone are shown in FIG. 4 and FIG. 5.
4) X-Ray Powder Diffraction: The diffraction patterns obtained with the complex and for the physical mixture of clarithromycin and zinc acetate are shown in FIG. 6.

The following examples are intended to further exemplify the inventions, but not to limit the scope of the invention:

### EXAMPLE 1

| Ingredients | Percentage weight (% w/w) |
|---|---|
| Clarithromycin | 1.03 |
| Butylated Hydroxy Anisole | 0.2 |
| Hydroxy Propyl Cellulose | 2.0 |
| Propylene Glycol | 20.0 |
| Zinc Acetate | 0.386 |
| Ethanol absolute | qs |

### PROCESS FOR PREPARING THE TOPICAL GEL COMPOSITION EXAMPLE 1

1) Zinc acetate was dissolved in propylene glycol.

2) Clarithromycin was dispersed in the bulk of the solution obtained in step 1.

3) Butylated hydroxy anisole was added to ethanol and this solution was blended with the solution of step 2 to form a clear solution.

4) Hydroxy propyl cellulose was then added under continuous stirring until the mixture was homogenized, while keeping the temperature at 25° C.

5) The gel was stirred at slow speed under vacuum and the weight was made up with ethanol.

The gel obtained according to the composition of Example 1 displayed the following properties:

| S. No. | Characteristics | Results |
|---|---|---|
| 1. | Physical | Colourless, transparent, viscous, homogenous |
| | appearance | gel |
| 2. | Viscosity* | 27,200 cps |
| 3. | Assay | 1.069% w/w |
| 4. | pH | 7.73 |

*(RVT model, spindle T-B, rpm-5)

The chemical and physical stability of packaged clarithromycin-zinc gel prepared according to the composition of Example 1, under accelerated stability conditions of 40±2° C. and 75±5% relative humidity, were evaluated on the basis of assay, viscosity and physical appearance measured between initial and three-month time points.

| Time | Parameters | | |
|---|---|---|---|
| (Months) | Assay (% | | |
| 40° C./75% RH | w/w) | Physical appearance | Viscosity (cps) |
| Initial | 1.069 | Colourless, transparent, | 27,220 |
| | | viscous, homogenous gel | |
| 1 Month | 1.050 | Colourless, transparent, | — |
| | | viscous, homogenous gel | |
| 2 Months | 1.047 | Colourless, transparent, | — |
| | | viscous, homogenous gel | |
| 3 Months | 0.995 | Colourless, transparent, | 26,600 |
| | | viscous, homogenous gel | |

### EXAMPLE 2

| Ingredients | Percentage weight (% w/w) |
|---|---|
| Clarithromycin | 1.03 |
| Butylated Hydroxy Anisole | 0.2 |
| Hydroxy Propyl Cellulose | 2.0 |
| Propylene Glycol | 20.0 |
| Zinc Acetate | 0.386 |
| Lactic acid | 0.25 |
| Ethanol (95%) qs to | q.s. |

### PROCESS FOR THE PREPARATION OF TOPICAL GEL COMPOSITION EXAMPLE 2

1) Zinc acetate was dissolved in propylene glycol

2) Lactic acid was dissolved in the solution obtained in step 1.

3) Clarithromycin was then dispersed in the solution obtained in step 2.

4) Butylated hydroxy anisole was added to ethanol and this solution was mixed with the solution of step 3 to form a clear solution.

5) Hydroxy propyl cellulose was then added under continuous stirring until the mixture was homogenized, while keeping the temperature at 25° C.

6) The gel was stirred at slow speed under vacuum and the weight was made up with ethanol.

The gel obtained according to the composition of Example 2 displayed the following properties:

| S. No. | Parameters | Results |
|---|---|---|
| 1. | Physical | Colourless, transparent, viscous, homogenous |
| | appearance | gel |
| 2. | Viscosity* | 28,800 cps |
| 3. | pH | 6.25 |
| 4. | Assay (% w/w) | 1.1014 |

*(RVT model, spindle T-B, rpm-5)

While several particular forms of the invention have been illustrated and described, it will be apparent that various modifications and combinations of the invention detailed in the text can be made without departing from the spirit and scope of the invention. Accordingly, it is not intended that the invention be limited, except as by the appended claims.

1. A transparent, topical composition comprising clarithromycin, a zinc salt, a pharmaceutically acceptable topical vehicle, and optionally one or more gelling agents.

2. The composition according to claim 1 wherein the clarithromycin comprises from about 0.1% w/w to about 10% w/w of the formulation.

3. The composition according to claim 1 wherein the zinc salt is selected from the group consisting of zinc acetate, zinc propionate, zinc butyrate, zinc pentanoate, zinc hexanoate, zinc heptanoate, zinc decanoate, zinc citrate, zinc maleate, zinc benzoate, zinc chloride, zinc sulfate, zinc phosphate, zinc bromide, zinc salts of amino acid, zinc alanine, zinc methionine and zinc glycine.

4. The composition according to claim 1 wherein the clarithromycin and the zinc salt are present in a molar ratio of about 1:0.2 to about 1:2.

5. The composition according to claim 4 wherein the clarithromycin and the zinc salt are combined in the molar ratio of about 1:1 to about 1:1.5.

6. The composition according to claim 1 wherein the topical vehicle comprises a non-aqueous or a hydroalcoholic material.

7. The composition according to claim 1 wherein the pharmaceutically acceptable topical vehicle comprises a non-aqueous material selected from one or more of the group of methanol, ethanol, n-propanol, isopropanol, butanol, propylene glycol, polypropylene glycol, polyethylene glycol, hydrocarbon oils and waxes, lanolin and lanolin derivatives, diisopropyl sebacate, isopropyl myristate, methyl laurate, silicon oil, glycerin, caprylic acid esters, transcutol, labrasol, labrafac, labrafil and mixtures thereof.

8. The composition according to claim 1 wherein the one or more gelling agent are selected from the group consisting of cellulose ethers, carbomers, hydroxypropyl cellulose, hydroxymethyl cellulose, hydroxypropyl methyl cellulose, carboxy methyl cellulose, sodium carboxy methyl cellulose, hydroxycellulose, vinyl alcohols, vinyl pyrrolidones, natural gums, karaya gum, locust bean gum, guar gum, gelan gum, xanthan gum, gum arabic, tragacanth gum, carrageenan, pectin, agar, alginic acid, sodium alginate, methacrylates, polyacrylates, and polyoxyethylene-polyoxypropylene copolymers.

9. The composition according to claim 1 wherein the composition is stable.

10. The composition according to claim 1 further comprising one or more pharmaceutically acceptable excipients, wherein the pharmaceutically acceptable excipients are selected from one or more of antioxidants, preservatives, pH modifying agent, perfumes, skin penetration enhancers and stabilizers.

11. The composition according to claim 10 wherein the pH modifying agent is selected from one or more of organic aliphatic polyhydroxy carboxylic acids, triethanolamine, diethanolamine, diethyl amine, sodium hydroxide, 2 amino-2 methyl-1 propanol, and tris buffer.

12. The composition according to claim 10 wherein the concentration of the pH modifying agent comprises between about 0.15-2% w/w of the final composition.

13. The composition according to claim 10 wherein the pH of the final composition is between about 6.0 and about 8.0.

14. The composition according to claim 10 wherein the skin penetration enhancers are selected from one or more of alcohols, short chain alcohols, polyalcohols, amino acids, fatty acids and their esters, azone and azone-like compounds, surfactants, and bile salts.

15. The composition according to claim 1 wherein the clarithromycin and the zinc salt form a clarithromycin zinc complex.

16. A process for the preparation of a topical composition of clarithromycin comprising the steps of:
a) dissolving a zinc salt in the presence of a suitable vehicle to form a solution;
b) dispersing clarithromycin in the solution;
c) mixing the solution to form a transparent solution; and, optionally
d) dispersing a gelling agent in the solution.

17. The process according to claim 16 wherein the clarithromycin and the zinc salt are combined in a molar ratio of about 1:0.2 to about 1:2.

18. The process according to claim 16 wherein the topical vehicle comprises a non aqueous or a hydroalcoholic material.

19. The process according to claim 16 further comprising the step of adding one or more pharmaceutically acceptable excipients selected from one or more of antioxidants, preservatives, pH modifying agent, perfumes, skin penetration enhancers and stabilizers.

20. The process according to claim 19 wherein the pH of the final composition is between about 6.0 and about 8.0.

21. The process according to claim 16 wherein the clarithromycin and the zinc salt form a clarithromycin zinc complex.

22. The process according to claim 21 wherein the clarithromycin, zinc salt, and excipients are all fully dissolved in the solution and the composition is colorless and stable.

23. A method of treating acne in a patient comprising administering a transparent topical composition comprising clarithromycin, a zinc salt, a pharmaceutically acceptable topical vehicle, and, optionally, one or more gelling agents.

24. The method of treating acne according to claim 23 wherein the clarithromycin and zinc salt form a complex.

25. The method of treating acne of claim 23 wherein the composition is colorless and stable.
